(19) United States
(12) Patent Application Publication
(10) Pub. No.: US 2011/0269778 A1
(43) Pub. Date: Nov. 3, 2011

(54) NOVEL DIPHENYL PURINE DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Glostrup (DK); Dan Peters, Malmo (DK)

(73) Assignee: NEUROSEARCH A/S, Ballerup (DK)

(21) Appl. No.: 13/123,259

(22) PCT Filed: Oct. 8, 2009
(86) PCT No.: PCT/EP2009/063102

§ 371 Date: May 24, 2011

Related U.S. Application Data
(60) Provisional application No. 61/104,324, filed on Oct. 10, 2008.

(30) Foreign Application Priority Data
Oct. 10, 2008 (DK) PA 2008 01424

(51) Int. Cl. A61K 31/52; C07D 487/04; A61K 31/522; A61K 31/519; A61P 25/22; A61P 25/00; A61P 25/28; A61P 25/16; A61P 25/24; A61P 25/18; A61P 3/04; A61P 25/14; A61P 25/08; A61P 11/06; A61P 17/00; A61P 25/06; A61P 25/30; A61P 1/00; C07D 473/00

(52) U.S. Cl. 514/261.1; 544/264; 544/277; 544/265; 544/254; 514/263.1; 514/263.3

(57) ABSTRACT

This invention relates to novel (6-phenyl-5-amino/nitro-pyrimidin-4-yl)-phenyl-amines, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel diphenyl purine derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

WO 99001439 describes aryl- and arylamino-substituted heterocyclic compounds useful as corticotropin releasing hormone antagonists. However, the diphenyl purine derivatives of the present invention, or their use as modulators of the nicotinic acetylcholine receptors, are not suggested.

Hauser et al. [Hauser D R J.; Scior T; Domeyer D M.; Kammerer B; Laufer S A; Journal of Medicinal Chemistry 2007 50 (9) 2060-2066] describes the synthesis, biological testing and binding mode prediction of 6,9-Diarylpurin-8-ones useful as p38 MAP Kinase Inhibitors. However, the diphenyl purine derivatives of the present invention, or their use as modulators of the nicotinic acetylcholine receptors, are not suggested.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides novel diphenyl purine derivative represented by Formula I

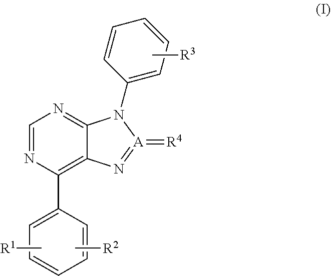

a stereoisomer thereof or a mixture of its stereoisomers, or a pharmaceutically acceptable salt thereof, wherein

N [symbol] A [symbol] R⁴ represents a conjugated system of double bonds;

A represents C or N;

one of R¹ and R² represents a substituent selected from the group consisting of halo, trifluoromethyl, trifluoromethoxy, nitro, cyano, hydroxy and alkoxy; and

the other of R¹ and R² represents a substituent selected from the group consisting of halo, trifluoromethyl, trifluoromethoxy, cyano, hydroxy and alkoxy; and

R³ represents hydrogen, hydroxy, alkoxy, sulfamoyl or pyrrolyl; and

R⁴ represents hydrogen, amino, nitro, hydroxy or oxy.

In a second aspect the invention provides pharmaceutical compositions comprising a therapeutically effective amount of the diphenyl purine derivative of the invention, or a pharmaceutically acceptable addition salt thereof, together with at least one pharmaceutically acceptable carrier or diluent.

Viewed from another aspect the invention relates to the use of the diphenyl purine derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the diphenyl purine derivative of the invention.

Other objects of the invention will be apparent to the person skilled in the art from the following detailed description and examples.

### DETAILED DISCLOSURE OF THE INVENTION

### Diphenyl Purine Derivatives

In its first aspect the invention provides novel diphenyl purine derivative represented by Formula I

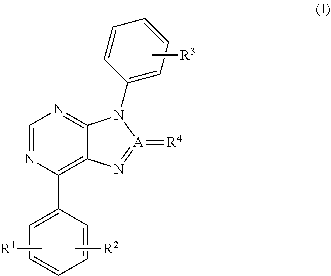

a stereoisomer thereof or a mixture of its stereoisomers, or a pharmaceutically acceptable salt thereof, wherein

N [symbol] A [symbol] R⁴ represents a conjugated system of double bonds;

A represents C or N;

one of R¹ and R² represents a substituent selected from the group consisting of halo, trifluoromethyl, trifluoromethoxy, nitro, cyano, hydroxy and alkoxy; and

the other of R¹ and R² represents a substituent selected from the group consisting of halo, trifluoromethyl, trifluoromethoxy, cyano, hydroxy and alkoxy; and

R³ represents hydrogen, hydroxy, alkoxy, sulfamoyl or pyrrolyl; and

R⁴ represents hydrogen, amino, nitro, hydroxy or oxy.

In a preferred embodiment the diphenyl purine derivative of the invention is a compound represented by Formula Ia

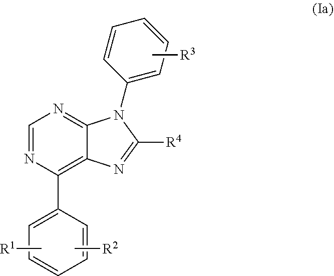

a stereoisomer thereof or a mixture of its stereoisomers, or a pharmaceutically acceptable salt thereof, wherein

R¹ and R², independently of each other, represent a substituent selected from the group consisting of halo, trifluoromethyl, trifluoromethoxy, nitro, cyano, hydroxy and alkoxy;

R³ represents hydrogen, hydroxy, alkoxy, sulfamoyl or pyrrolyl; and

R⁴ represents hydrogen, amino, nitro or hydroxy.

In another preferred embodiment the diphenyl purine derivative of the invention is a compound represented by Formula Ib

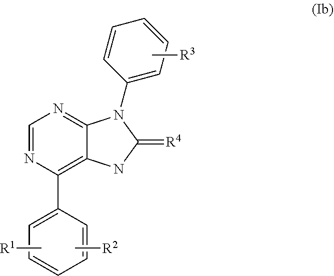

a stereoisomer thereof or a mixture of its stereoisomers, or a pharmaceutically acceptable salt thereof, wherein

R¹ and R², independently of each other, represent a substituent selected from the group consisting of halo, trifluoromethyl, trifluoromethoxy, nitro, cyano, hydroxy and alkoxy;

R³ represents hydrogen, hydroxy, alkoxy, sulfamoyl or pyrrolyl; and

R⁴ represents oxy.

In a third preferred embodiment the diphenyl purine derivative of the invention is a diphenyl triazolo-pyrimidine derivative represented by Formula Ic

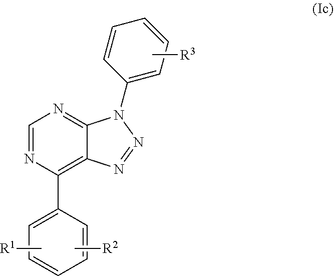

a stereoisomer thereof or a mixture of its stereoisomers, or a pharmaceutically acceptable salt thereof, wherein

one of R¹ and R² represents a substituent selected from the group consisting of halo, trifluoromethyl, trifluoromethoxy, nitro, cyano, hydroxy and alkoxy; and

the other of R¹ and R² represents a substituent selected from the group consisting of halo, trifluoromethyl, trifluoromethoxy, cyano, hydroxy and alkoxy; and

R³ represents hydrogen, hydroxy, alkoxy, sulfamoyl or pyrrolyl.

In a more preferred embodiment the diphenyl purine derivative of the invention is a compound represented by Formula I, Ia or Ib, wherein R¹ and R², independently of each other, represent a substituent selected from the group consisting of halo, trifluoromethyl, trifluoromethoxy, nitro, cyano, hydroxy and alkoxy.

In another more preferred embodiment the diphenyl purine derivative of the invention is a compound represented by Formula I, Ia or Ib, wherein R¹ and R², independently of each other, represent a substituent selected from the group consisting of halo, trifluoromethyl, trifluoromethoxy and nitro.

In a third more preferred embodiment the diphenyl purine derivative of the invention is a compound represented by Formula I, Ia or Ib, wherein R¹ and R², independently of each other, represent a substituent selected from the group consisting of halo and trifluoromethyl.

In a fourth more preferred embodiment the diphenyl purine derivative of the invention is a compound represented by Formula I, Ia or Ib, wherein R¹ and R², independently of each other, represent a substituent selected from the group consisting of fluoro and trifluoromethyl.

In a fifth more preferred embodiment the diphenyl purine derivative of the invention is a compound represented by Formula I, Ia or Ib, wherein one of R¹ and R² represents halo, and in particular fluoro; and the other of R¹ and R² represents trifluoromethyl.

In a fourth preferred embodiment the compound of the invention is a diphenyl triazolo-pyrimidine derivative of Formula Ic, wherein one of R¹ and R² represents a substituent selected from the group consisting of halo, trifluoromethyl, trifluoromethoxy, nitro, cyano, hydroxy and alkoxy; and the other of R¹ and R² represents a substituent selected from the group consisting of halo, trifluoromethyl, trifluoromethoxy, cyano, hydroxy and alkoxy.

In a more preferred embodiment the compound of the invention is a diphenyl triazolo-pyrimidine derivative of Formula Ic, wherein R¹ and R², independently of each other, represent a substituent selected from the group consisting of halo, trifluoromethyl, trifluoromethoxy, cyano, hydroxy and alkoxy.

In another more preferred embodiment the compound of the invention is a diphenyl triazolo-pyrimidine derivative of Formula Ic, wherein R¹ and R², independently of each other, represent a substituent selected from the group consisting of halo, trifluoromethyl, trifluoromethoxy and nitro.

In a third more preferred embodiment the compound of the invention is a diphenyl triazolo-pyrimidine derivative of Formula Ic, wherein R¹ and R², independently of each other, represent a substituent selected from the group consisting of halo and trifluoromethyl.

In a fourth more preferred embodiment the compound of the invention is a diphenyl triazolo-pyrimidine derivative of Formula Ic, wherein one of R¹ and R² represents halo, and in particular fluoro; and the other of R¹ and R² represents trifluoromethyl.

In a fifth preferred embodiment the diphenyl purine derivative of the invention is a compound represented by Formula I, Ia or Ib, wherein R³ represents hydrogen, hydroxy, alkoxy, sulfamoyl or pyrrolyl.

In a more preferred embodiment R³ represents hydroxy, alkoxy, sulfamoyl or pyrrolyl.

In another more preferred embodiment R³ represents hydroxy, alkoxy or sulfamoyl.

In a third more preferred embodiment R³ represents hydroxy.

In a fourth more preferred embodiment, R³ represents alkoxy, and in particular methoxy.

In a fifth more preferred embodiment R³ represents sulfamoyl.

In a sixth preferred embodiment the diphenyl purine derivative of the invention is a compound represented by Formula I, Ia or Ib, wherein R⁴ represents hydrogen, amino, nitro, hydroxy or oxy.

In a more preferred embodiment the diphenyl purine derivative of the invention is a compound represented by Formula Ia, wherein R⁴ represents hydrogen, amino or nitro.

In another more preferred embodiment the diphenyl purine derivative of the invention is a compound represented by Formula Ia, wherein R⁴ represents hydrogen.

In a third more preferred embodiment the diphenyl purine derivative of the invention is a compound represented by Formula Ia, wherein R⁴ represents amino.

In a fourth more preferred embodiment the diphenyl purine derivative of the invention is a compound represented by Formula Ia, wherein R⁴ represents nitro.

In a fifth more preferred embodiment the diphenyl purine derivative of the invention is a compound represented by Formula Ib, wherein R⁴ represents oxy.

In a sixth more preferred embodiment the compound of the invention is a diphenyl triazolo-pyrimidine derivative of Formula Ic, wherein R³ represents hydroxy, alkoxy or sulfamoyl.

In a seventh more preferred embodiment the compound of the invention is a diphenyl triazolo-pyrimidine derivative of Formula Ic, wherein R³ represents hydroxy or alkoxy, and in particular methoxy.

In an eight more preferred embodiment the compound of the invention is a diphenyl triazolo-pyrimidine derivative of Formula Ic, wherein R³ represents hydroxy.

In a ninth more preferred embodiment the compound of the invention is a diphenyl triazolo-pyrimidine derivative of Formula Ic, wherein R³ represents alkoxy, and in particular methoxy.

In a most preferred embodiment the compound of the invention is
6-(2-Fluoro-4-trifluoromethyl-phenyl)-9-(4-methoxy-phenyl)-9H-purine;
4-[6-(2-Fluoro-4-trifluoromethyl-phenyl)-purin-9-yl]-phenol;
4-[6-(2-Fluoro-4-trifluoromethyl-phenyl)-purin-9-yl]-benzenesulfonamide;
6-(2-Fluoro-4-trifluoromethyl-phenyl)-9-(4-methoxy-phenyl)-7,9-dihydro-purin-8-one;
6-(2-Fluoro-4-trifluoromethyl-phenyl)-9-(4-hydroxy-phenyl)-7,9-dihydro-purin-8-one;
7-(2-Fluoro-4-trifluoromethyl-phenyl)-3-(4-methoxy-phenyl)-3H-[1,2,3]triazolo[4,5-d]pyrimidine; or
4-[7-(2-Fluoro-4-trifluoromethyl-phenyl)-[1,2,3]triazolo[4,5-d]pyrimidin-3-yl]-phenol;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

### Pharmaceutically Acceptable Salts

The diphenyl purine derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a diphenyl purine derivative of the invention include alkali metal salts, such as the sodium salt of a compound of the invention containing a carboxy group.

### Steric Isomers

It will be appreciated by those skilled in the art that the diphenyl purine derivatives of the present invention may exist in different stereo isomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Producing Diphenyl Purine Derivatives

The diphenyl purine derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with drawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of diphenyl purine derivative of the invention.

While a diphenyl purine derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the diphenyl purine derivative of the invention, or a pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The diphenyl purine derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a diphenyl purine derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

### General Synthetic Procedure for the Compounds of the Invention

Nucleophilic substitution of a commercial 4,6-dihalo-5-nitropyrimidine with the appropriate commercial aniline in boiling 2-propanol yielded the correspondent phenyl-pyrimidin-4-yl-amines (A), as described by Clark et al. [Clark R L, Pessolano A A, Shen T Y, Jacobus D P, Jones H, Lotti V J, Flataker L M; Journal of Medicinal Chemistry 1978 21 (9) 965-978]. Suzuki coupling reaction of these latter compounds with the suitably-substituted benzeneboronic acids afforded the (6-phenyl-5-nitro-pyrimidin-4-yl)-phenyl-amines (B), as described by Hauser et al. [Hauser D R J, Scior T, Domeyer D M, Kammerer B, Laufer S A; Journal of Medicinal Chemistry 2007 50 (9) 2060-2066], which were then catalytically hydrogenated to give the correspondent (6-phenyl-5-amino-pyrimidin-4-yl)-phenyl-amines (C). These latter diamines were cyclized in different conditions, to afford the compounds of the invention (D). Those cyclic derivatives D bearing a methoxy substituent in R3 were further subjected to ether hydrolysis upon mild nucleophilic substitution with the Lewis acid boron tribromide (E).

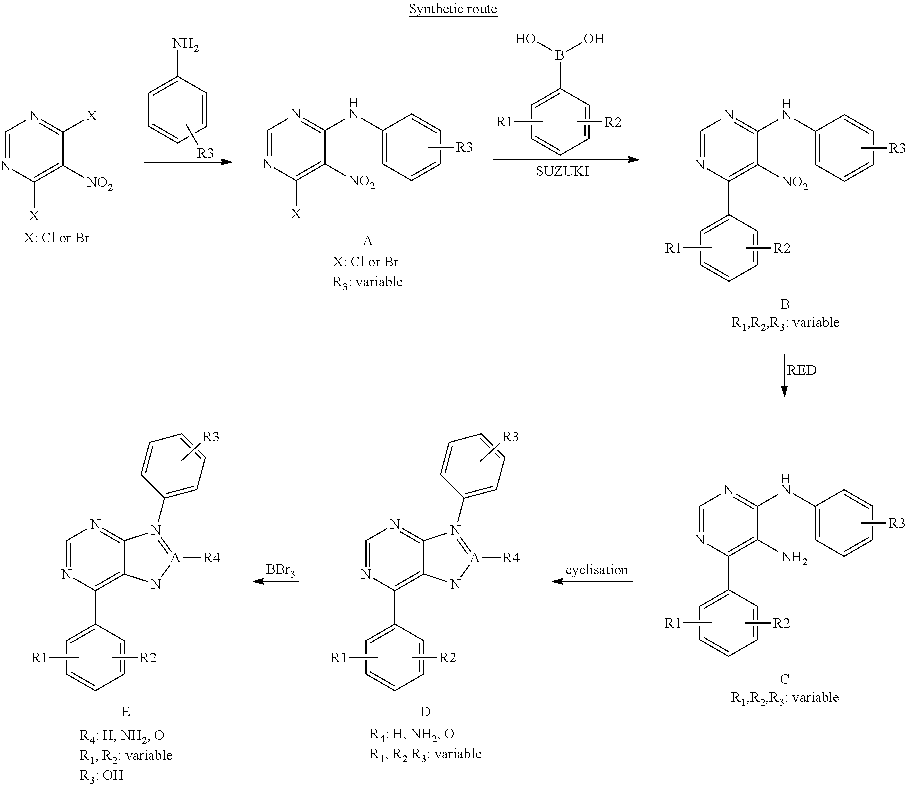

(6-Chloro-5-nitro-pyrimidin-4-yl)-(4-methoxy-phenyl)-amine (Intermediate compound INT-1)

To a stirred and ice-cooled solution of 4,6-dichloro-5-nitropyrimidine (1.000 g, 5.1553 mmol, 1 eq) in anhydrous 2-propanol (10 ml), 4-methoxy-phenylamine (0.6984 g, 5.6708 mmol, 1.1 eq) and triethylamine (1.304 g, 12.888 mmol, 2.5 eq) were added drop-wise and the mixture refluxed under a nitrogen atmosphere for 2 hours. The resulting reaction mixture was evaporated, and the solid residue was suspended in water (100 ml) and extracted with chloroform (150 ml×3). The combined organic layers were washed with brine, dried over anhydrous sodium sulphate, filtered and evaporated, to afford the title compound (˜1.400 g, 96% mass balance) as a red solid. This latter was purified by column chromatography over silica gel eluting with 5% ethylacetate in hexane (0.600 g, 41% yield; MH+ 281, 96% pure at LCMS).

### [6-(2-Fluoro-4-trifluoromethyl-phenyl)-5-nitro-pyrimidin-4-yl]-(4-methoxy-phenyl)-amine (Intermediate compound INT-2)

A mixture of (6-chloro-5-nitro-pyrimidin-4-yl)-(4-methoxy-phenyl)-amine (INT-1; 0.500 g, 1.7815 mmol, 1 eq), 2-fluoro-4-(trifluoromethyl)phenylboronic acid (0.4075 g, 1.9597 mmol, 1.1 eq), sodium carbonate (0.3776 g, 3.563 mmol, 2 eq) and 1,4-dioxane (10 ml) was degassed with nitrogen and kept under a nitrogen atmosphere during the entire course of the reaction. To the degassed mixture, palladium (II) (bistriphenylphosphine)dichloride (0.0625 g, 0.0891 mmol, 0.05 eq) was added and the resulting reaction mixture, refluxed overnight and cooled to room temperature, was worked up by evaporation to dryness followed by addition of water and finally extracted with chloroform. The combined organic layers, dried over anhydrous MgSO₄, afforded upon evaporation a red solid material (0.600 g), which eluted over silica gel (60-120 mesh) with 7% ethylacetate in hexane gave 0.220 g (30% yield) of the pure title compound as an orange solid. M.p. 155.6-155.8° C. LC-ESI-HRMS of [M+H]+shows 409.0904 Da. Calc. 409.092378 Da, dev. −4.8 ppm.

### 6-(2-Fluoro-4-trifluoromethyl-phenyl)-N*4*-(4-methoxy-phenyl)-pyrimidine-4,5-diamine (Intermediate compound INT-3)

A degassed mixture of a solution of [6-(2-fluoro-4-trifluoromethyl-phenyl)-5-nitro-pyrimidin-4-yl]-(4-methoxy-phenyl)-amine (INT-2; 1.000 g, 2.4491 mmol, 1 eq) in methanol (10 ml) and raney-nickel (0.100 g, ˜0.3 eq) was put under a hydrogen atmosphere and stirred at room temperature for 4 hours. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.800 g (86% yield) of the title compound as a white solid, which is 99% pure at HPLC. M.p. 212.1-213.2° C. LC-ESI-HRMS of [M+H]+shows 379.1172 Da. Calc. 379.118198 Da, dev. −2.6 ppm.

### 4-(6-Chloro-5-nitro-pyrimidin-4-ylamino)-benzenesulfonamide (Intermediate compound INT-4)

To a stirred and ice-cooled solution of 4,6-dichloro-5-nitropyrimidine (3.000 g, 15.4658 mmol, 1 eq) in anhydrous 2-propanol (30 ml), 4-amino-benzenesulfonamide (2.718 g, 15.4658 mmol, 1.1 eq) and triethylamine (3.130 g, 30.9316 mmol, 2 eq) were added drop-wise and the mixture refluxed under a nitrogen atmosphere for 3 hours. The resulting reaction mixture was evaporated, and the solid residue was suspended in water (150 ml) and extracted with ethylacetate (200 ml×3). The combined organic layers were washed with brine, dried over anhydrous sodium sulphate, filtered and evaporated, to afford the title compound (˜4.500 g, 89% mass balance) as a yellow solid. This latter was purified by column chromatography over silica gel eluting with 15% ethylacetate in hexane (1.400 g, 27% yield; MH+ 330, 93% pure at LCMS).

### 4-[6-(2-Fluoro-4-trifluoromethyl-phenyl]-5-nitro-pyrimidin-4-ylamino]-benzenesulfonamide (Intermediate compound INT-5)

To a degassed mixture of 4-(6-chloro-5-nitro-pyrimidin-4-ylamino)-benzenesulfonamide (INT-4; 1.400 g, 4.246 mmol, 1 eq), 2-fluoro-4-(trifluoromethyl)phenylboronic acid (0.9711 g, 4.6706 mmol, 1.1 eq), sodium carbonate (1.125 g, 10.615 mmol, 2.5 eq), 1,4-dioxane (15 ml) and water (5 ml), palladium (II) (bistriphenylphosphine)dichloride (0.149 g, 0.2123 mmol, 0.05 eq) was added and the resulting reaction mixture, refluxed for 5 hours and cooled to room temperature, was worked up by concentration under reduced pressure followed by addition of water and finally extracted with chloroform. The combined organic layers, dried over anhydrous MgSO₄, afforded upon evaporation a yellow solid material (1.700 g, 87% mass balance), which eluted over silica gel (230-400 mesh) with 12% ethylacetate in hexane gave 0.503 g (32% yield) of the pure title compound as an orange solid. MH+ 458.

### 4-[5-Amino-6-(2-fluoro-4-trifluoromethyl-phenyl)-pyrimidin-4-ylamino]-benzenesulfonamide (Intermediate compound INT-6)

A degassed mixture of a solution of 4-[6-(2-fluoro-4-trifluoromethyl-phenyl)-5-nitro-pyrimidin-4-ylamino]-benzenesulfonamide (INT-5; 0.900 g, 1.9678 mmol, 1 eq) in methanol (15 ml) and raney-nickel (0.080 g, ˜0.3 eq) was put under a hydrogen atmosphere and stirred at room temperature overnight. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.700 g (77% yield) of the title compound as a white solid. After washing with diethylether, the residual solid (0.402 g, 41% yield) resulted to be 96% pure at HPLC.

### 6-(2-Fluoro-4-trifluoromethyl-phenyl)-9-(4-methoxy-phenyl)-9H-purine (Compound 1)

To a stirred solution of 6-(2-fluoro-4-trifluoromethyl-phenyl)-N*4*-(4-methoxy-phenyl)-pyrimidine-4,5-diamine (INT-3; 1.000 g, 2.6432 mmol) and p-toluenesulphonic acid (0.3000 g, 1.5771 mmol) in anhydrous tetrahydrofuran (15 ml), trimethyl orthoformate (0.4207 g, 3.9648 mmol) was added drop-wise and heated at 70° C. for 6 h. The reaction mixture was quenched by careful addition of saturated aqueous sodium bicarbonate (−50 ml), and extracted with chloroform (250 ml×3). The combined organic layers, washed with brine, dried over anhydrous sodium sulphate, filtered and evaporated, to afford a crude material (0.9500 g, 92% mass balance). This was purified by column chromatography over silica gel (230-400 mesh) eluting with 14% ethylacetate in hexane, to furnish the title compound (0.651 g, 63% yield) as a pink solid. M.p. 181.4-182.8° C. LC-ESI-HRMS of [M+H]+shows 389.1031 Da. Calc. 389.102548 Da, dev. 1.4 ppm.

### 4-[6-(2-Fluoro-4-trifluoromethyl-phenyl)-purin-9-yl]-phenol (Compound 2)

To a solution of 6-(2-fluoro-4-trifluoromethyl-phenyl)-9-(4-methoxy-phenyl)-9H-purine (Compound 1; 0.400 g, 1.0301 mmol) in anhydrous dichloromethane (10 ml), cooled to −78° C. and kept under nitrogen, a solution of boron tribromide (1.2903 g, ˜0.5 ml, 5.1505 mmol) in anhydrous dichloromethane (5 ml) was added drop-wise. The reaction mixture was allowed to attain room temperature spontaneously and stirred overnight. The mixture was then cooled again in an ice-salt bath and the excess of the reagent was decomposed by treatment with methanol (5 ml) followed by water (10 ml) and finally extracted with chloroform. The combined organic layers, dried over anhydrous MgSO₄, afforded upon evaporation the title compound as a white solid material (˜0.380 g), which was purified by crystallization from a mixture of dichloromethane and hexane (0.358 g, 92% yield). M.p. 205.2-206.4° C.

### 4-[6-(2-Fluoro-4-trifluoromethyl-phenyl)-purin-9-yl]-benzenesulfonamide (Compound 3)

To a stirred solution of 4-[5-amino-6-(2-fluoro-4-trifluoromethyl-phenyl)-pyrimidin-4-ylamino]-benzenesulfonamide (INT-6; 0.300 g, 0.702 mmol) and p-toluenesulphonic acid (0.1335 g, 0.702 mmol) in 1,2-dichloroethane (5 ml), trimethyl orthoformate (0.1117 g, 1.053 mmol) was added drop-wise and heated at 70° C. for 3 h. The reaction mixture was quenched by careful addition of saturated aqueous sodium bicarbonate, and extracted with chloroform. The combined organic layers, washed with brine, dried over anhydrous sodium sulphate, filtered and evaporated, to afford a crude material. This was purified by column chromatography over silica gel (230-400 mesh) eluting with 23% ethylacetate in hexane, to furnish the title compound (0.221 g, 72% yield) as an off-white solid. M.p. 243.8-245° C.

### 6-(2-Fluoro-4-trifluoromethyl-phenyl)-9-(4-methoxy-phenyl)-7,9-dihydro-purin-8-one (Compound 4)

To an ice-cooled and stirred solution of 6-(2-fluoro-4-trifluoromethyl-phenyl)-N*4*-(4-methoxy-phenyl)-pyrimidine-4,5-diamine (INT-3; 0.900, 2.3789 mmol) in anhydrous tetrahydrofuran (15 ml), triphosgene (0.7059 g, 2.3789 mmol) and triethylamine (0.481 g, ˜0.66 ml, 4.7578 mmol) were added drop-wise. The resulting reaction mixture was allowed to attain room temperature spontaneously, refluxed for 12 hours and evaporated. Water (−200 ml) was added and extraction with chloroform (250 ml×3) followed. The combined organic layers were washed with brine and dried over anhydrous sodium sulphate, filtered and evaporated, to afford a crude material (0.902 g, mass balance 93%). This was purified by column chromatography over silica gel (230-400 mesh) eluting with 16% ethyl acetate in hexane, to furnish the title compound as a white solid (0.650 g, 58% yield). M.p. 295.9-297.2° C.

### 6-(2-Fluoro-4-trifluoromethyl-phenyl)-9-(4-hydroxy-phenyl)-7,9-dihydro-purin-8-one (Compound 5)

To a solution of 6-(2-fluoro-4-trifluoromethyl-phenyl)-9-(4-methoxy-phenyl)-7,9-dihydro-purin-8-one (Compound 4; 0.370 g, 0.9151 mmol) in anhydrous dichloromethane (10 ml), cooled to −78° C. and kept under nitrogen, a solution of boron tribromide (1.146 g, ˜0.4 ml, 4.5755 mmol) in anhydrous dichloromethane (5 ml) was added drop-wise. The reaction mixture was allowed to attain room temperature spontaneously and stirred overnight. The mixture was then cooled again in an ice-salt bath and the excess of the reagent was decomposed by treatment with methanol (5 ml) followed by water (10 ml) and finally extracted with chloroform. The combined organic layers, dried over anhydrous MgSO₄, afforded upon evaporation the title compound as a white solid material (˜0.250 g), which was purified by column chromatography over silica gel (230-400 mesh) eluting with 30% ethylacetate in hexane (0.110 mg, 30% yield). M.p. 276.4-277.9° C.

### 7-(2-Fluoro-4-trifluoromethyl-phenyl)-3-(4-methoxy-phenyl)-3H-[1,2,3]-triazolo[4,5-d]pyrimidine (Compound 6)

To a stirred solution of 6-(2-fluoro-4-trifluoromethyl-phenyl)-N*4*-(4-methoxy-phenyl)-pyrimidine-4,5-diamine (INT-3; 3.200 g, 8.4583 mmol) in 50% aqueous acetic acid (40 ml) and dichloromethane (40 ml), sodium nitrite (0.642 g, 9.3041 mmol) in water (5 ml) was added drop-wise at room temperature. After addition, the reaction was stirred for 3 hours and the organic layer was then separated. This latter was washed with brine, dried over MgSO₄, filtered and evaporated, to give 3.100 g (94% mass balance) of a white solid, which was purified by crystallization from a mixture of dichloromethane and petroleum ether (2.010 g, ˜60% yield). M.p. 175.4-176.8° C.

4-[7-(2-Fluoro-4-trifluoromethyl-phenyl)-[1,2,3]triazolo[4,5-d]pyrimidin-3-yl]-phenol (Compound 7)

To a solution of 7-(2-fluoro-4-trifluoromethyl-phenyl)-3-(4-methoxy-phenyl)-3H-[1,2,3]triazolo[4,5-d]pyrimidine (Compound 6; 0.800 g, 2.0549 mmol) in anhydrous dichloromethane (20 ml), cooled to −78° C. and kept under nitrogen, a solution of boron tribromide (3.089 g, ˜1.2 ml, 12.3294 mmol) in anhydrous dichloromethane (8 ml) was added drop-wise. The reaction mixture was allowed to attain room temperature spontaneously and stirred overnight. The mixture was then cooled again in an ice-salt bath and the excess of the reagent was decomposed by treatment with methanol (30 ml) followed by water (30 ml) and finally extracted with chloroform. The combined organic layers, dried over anhydrous MgSO₄, afforded upon evaporation the title compound as a white solid material, which was purified by column chromatography over silica gel (230-400 mesh) eluting with 10% ethylacetate in hexane (0.370 mg, 47% yield). M.p. 210.2-211.8° C.

### Example 2

### Biological Activity

In this example the positive modulation of wild-type nAChR α7 receptors by the compounds of the invention was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent test applications, increasing concentrations (0.01-31.6 μM) of the test compound were applied 30 s before and during the acetylcholine (100 μM) application, which caused a robust increase in the acetylcholine-induced current amplitude.

The positive modulation in the presence of Compound 4 was calculated as (test-control)/control×100% and the concentration response curve for this positive modulation was fitted to the sigmoidal logistic equation: I=Iₘₐₓ/(1+(EC₅₀/[compound])ⁿ), where Iₘₐₓ represents the maximal modulation of the control response, EC₅₀ is the concentration causing a half maximal response, and n is the slope coefficient.

Calculated EC₅₀ and EC₅₀ Iₘₐₓ values are presented in the table below.

This is an indication of a biological activity as modulators of the nicotinic acetylcholine α7 receptor subtype.

| Compound | EC₅₀ (μM) | EC₅₀ Iₘₐₓ (%) |
|---|---|---|
| Compound 1 | 1.2 | 45 |
| Compound 2 | 5.8 | 120 |
| Compound 3 | 9.5 | 44 |
| Compound 4 | 3.2 | 1.6 |
| Compound 5 | 33 | 82 |
| (Compound 6) | — | 7.3 |
| (Compound 7) | 5.9 | 28 |

1. A diphenyl purine derivative represented by Formula I 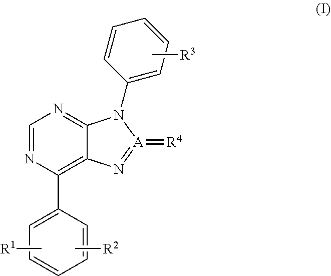
a stereoisomer thereof or a mixture of its stereoisomers, or a pharmaceutically acceptable salt thereof, wherein
N [symbol] A [symbol] R⁴ represents a conjugated system of double bonds;
A represents C or N;
one of R¹ and R² represents a substituent selected from the group consisting of halo, trifluoromethyl, trifluoromethoxy, nitro, cyano, hydroxy and alkoxy; and
the other of R¹ and R² represents a substituent selected from the group consisting of halo, trifluoromethyl, trifluoromethoxy, cyano, hydroxy and alkoxy; and
R³ represents hydrogen, hydroxy, alkoxy, sulfamoyl or pyrrolyl; and
R⁴ represents hydrogen, amino, nitro, hydroxy or oxy.

2. The diphenyl purine derivative of claim 1, a stereoisomer thereof or a mixture of its stereoisomers, or a pharmaceutically acceptable salt thereof, wherein
R¹ and R², independently of each other, represent a substituent selected from the group consisting of halo, trifluoromethyl, trifluoromethoxy, nitro, cyano, hydroxy and alkoxy.

3. The diphenyl purine derivative of claim 1, a stereoisomer thereof or a mixture of its stereoisomers, or a pharmaceutically acceptable salt thereof, wherein
R³ represents hydrogen, hydroxy, alkoxy, sulfamoyl or pyrrolyl.

4. The diphenyl purine derivative of claim 1, a stereoisomer thereof or a mixture of its stereoisomers, or a pharmaceutically acceptable salt thereof, wherein
R⁴ represents hydrogen, amino, nitro, hydroxy or oxy.

5. The diphenyl purine derivative derivative of claim 1, which is
6-(2-Fluoro-4-trifluoromethyl-phenyl)-9-(4-methoxy-phenyl)-9H-purine;
4-[6-(2-Fluoro-4-trifluoromethyl-phenyl)-purin-9-yl]-phenol;
4-[6-(2-Fluoro-4-trifluoromethyl-phenyl)-purin-9-yl]-benzenesulfonamide;
6-(2-Fluoro-4-trifluoromethyl-phenyl)-9-(4-methoxy-phenyl)-7,9-dihydro-purin-8-one;
6-(2-Fluoro-4-trifluoromethyl-phenyl)-9-(4-hydroxy-phenyl)-7,9-dihydro-purin-8-one;
7-(2-Fluoro-4-trifluoromethyl-phenyl)-3-(4-methoxy-phenyl)-3H-[1,2,3]triazolo[4,5-d]pyrimidine; or
4-[7-(2-Fluoro-4-trifluoromethyl-phenyl)-[1,2,3]triazolo[4,5-d]pyrimidin-3-yl]-phenol;
a stereoisomer thereof or a mixture of its stereoisomers, or a pharmaceutically acceptable salt thereof.

6. A pharmaceutical composition comprising a therapeutically effective amount of a diphenyl purine derivative of claim 1, a stereoisomer thereof or a mixture of its stereoisomers, or a pharmaceutically acceptable addition salt thereof, together with at least one pharmaceutically acceptable carrier or diluent.

7. The diphenyl purine derivative derivative of claim 1, a stereoisomer thereof or a mixture of its stereoisomers, or a pharmaceutically acceptable addition salt thereof, for use as a medicament.

8. (canceled)

9. The method according to claim 10, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with drawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.

10. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a diphenyl purine derivative of claim 1, a stereoisomer thereof or a mixture of its stereoisomers, or a pharmaceutically acceptable salt thereof.
